(19) United States
(12) Patent Application Publication
(10) Pub. No.: US 2008/0026472 A1
(43) Pub. Date: Jan. 31, 2008

(54) Instrument For Efficient Treatment Of Analytical Devices

(76) Inventors: Carsten Haack, Cham (CH); Ueli Stettler, Cham (CH)

Correspondence Address: ROCHE MOLECULAR SYSTEMS INC;PATENT LAW DEPARTMENT, 1145 ATLANTIC AVENUE, ALAMEDA, CA, 94501, US

(21) Appl. No.: 11/629,250

(22) PCT Filed: Jul. 2, 2005
(86) PCT No.: PCT/EP05/07150

§ 371 Date: May 23, 2007

(30) Foreign Application Priority Data
Jul. 2, 2004 (EP) 04015574.9

(51) Int. Cl. C12Q 1/68; G01N 35/00

(52) U.S. Cl. 436/48; 422/63; 422/67

(57) ABSTRACT

Subject of the invention is an instrument for automatically processing analytical devices wherein efficient washing and hybridization is effected by moving the disposable in a controlled rotational way in heatable, open carrier manifolds.

Subject of the present invention is an instrument for processing one or more analytical devices containing immobilized binding reagents, a system comprising such instrument, a method for processing the analytical device and a method for the determination of an analyte in said device.

### BACKGROUND OF THE INVENTION

The invention is useful in the field of analytics, wherever a device containing immobilized reagents bound to an inner surface of said device are to be contacted with a sample to bind a component of said sample to said device. Particularly, the invention is useful in the field of diagnostics, particularly Molecular Diagnostics, e.g. the analysis of nucleic acid components or proteins in samples such as human body fluids or in environmental samples.

Due to the progress achieved in increasing the sensitivity of assays by amplifying nucleic acid sequences, for instance by the Polymerase Chain Reaction (PCR), as disclosed in EP 0 201 184 and subsequent detection as disclosed in EP 0 200 362, molecular diagnostics has been established as a tool to determine nucleic acid containing parameters, like viruses and bacteria, for instance Hepatitis B virus and HIV. PCR based assays were developed using the so called heterogeneous format as disclosed in EP 0 420 260. In those assays, exemplified in Roche's AMPLICOR assays, nucleic acid sequences of a nucleic acid of a defined analyte, like Hepatitis B virus, are amplified and immobilized on so called capture probes contained in a tube. Due to the slow diffusion of nucleic acids to the capture probes, the immobilization requires some time to come to completion. This disadvantage was avoided by the so called homogenous assays that do not need immobilized probes for the detection. An exemplary homogeneous assay method is disclosed in EP 0 543 942.

Instruments for performing PCR were developed to conveniently perform the required thermal cycles, also called thermo cycles, needed to anneal the primers to the target nucleic acid, extend the primers using the target nucleic acid as a template, and separate the nucleic acid strands to provide single strands that can again bind the primers. A thermocycler useful to conduct thermo cycles is disclosed in EP 0 236 069.

Due to the capacity of PCR to amplify nucleic acid sequences which are present in samples in only minute amounts and to amplify different sequences in one sample, assays were developed to amplify and detect several analytes or parameters independently in parallel. Particularly, if more then ten analytes are suspected to be contained and detected in one sample, those assays require the use of a corresponding number of probes, preferably immobilized to separate sites of a solid surface. The manufacture of chips containing a large number of different binding agents on a planar surface is disclosed in EP 0 476 014.

A device for holding chips and conducting analytical reactions in said device are proposed in EP 1 161 989. A first method for processing liquids in said device is disclosed in EP 1 226 863. In this method, a cartridge containing a chip is moved back and forth to mix the liquid contained in said cartridge. In EP 1 224 976 there is described a method for mixing a liquid in a cartridge wherein the cartridge is swung back and forth to force some liquid to pass the surface of the chip. Those devices have very thin cavities in order to avoid transport of liquid from large distances to the surface of the chip. Thin cavities have the disadvantage that filling with liquid requires relatively complicated inlet and outlet channels as well as special adapters to connect the inlet and outlet channel to a fluid system. The adapters bear the risk of leakage and/or damage and require accurate positioning of the cartridge.

In EP 0 695 941 there is disclosed a flat device containing a chip having a flat cavity, inlet and outlet channels being arranged on the flat surface of the device. Again, the device is difficult to fill because the inlet and outlet channels need to be connected tightly to the instrument, particularly, because the adapters to connect the cavity to the fluid system in the instrument need to be very accurate. U.S. Pat. No. 6,043,080 describes a flat device containing a chip. This device again suffers from the same disadvantages.

Another device for holding chips is disclosed in EP 1 419 821. Because this device has a thicker cavity, it can retain larger amounts of liquid. Diffusion of components of the liquid sample contained therein to the active surface is facilitated by vortexing the liquid sample for mixing.

In US 2004/0191807 and US 2004/0114456 instruments are disclosed comprising a housing containing cartridges including planar array chips wherein mixing is performed by rotating the housing around a rotational axis perpendicular to the chip plain in the range of 30 rpm up to 90 rpm, which is inefficient mixing and results in longer hybridization times. Another disadvantage of the described instrument is the arrangement of a hybridization unit as described in U.S. Pat. No. 6,050,719 and a fluidics or wash unit as described in U.S. Pat. No. 6,114,122 on a workbench served by an industrial handling robot, which needs a lot of space and is inefficient in timing. The use of fluid delivery systems with complex fluid connectors is also limiting an automated high throughput application.

In U.S. Pat. No. 6,660,233 there is disclosed an instrument involving transport of a substrate on bioarray chips mounted on a holder from a reaction station to a detection station. The holder with the attached array chips is immersed in a well filled with sample. However, the process performed on this instrument is not including typical wash steps or typical fluid applications for array chips. This limits the field of application and flexibility. In addition the splitting of chip holder and reaction vessel exposes the unprotected array chips. Another disadvantage is the non-continuous traceability of results if array chips are not connected with respective reaction vessels.

In U.S. Pat. No. 5,538,849 there is disclosed transporting of racks containing several vessels at once through an instrument. This is inflexible. The reaction vessels do not include array chips and the process described is not considering requirements connected to array chips applications.

In U.S. Pat. No. 5,215,714 there is a disclosure on hybridization and mixing on a reversibly rotatable rotor. The methods and reaction vessels disclosed there do not include array chips. In addition mixing on such a rotor is inefficient.

The instruments presently known have the disadvantage that they do not allow convenient and rapid processing of chip containing devices, particularly regarding assay format flexibility, automated docked workflow, reliable device handling, chip protection, precise chip positioning, ease of use and instrument loading. Existing instruments are limited in using a clocked processing and they are focusing on special applications.

The present invention is directed to improved handling and control of micro array- or chip-based test procedures in integrated instruments.

### BRIEF DESCRIPTION OF THE DRAWINGS

In FIG. 1 there is shown a device containing a chip having immobilized reagents on its surface.

FIG. 2 shows the treatment station according to the invention.

FIG. 3 shows the treatment station with washing unit for stationary wash dilution of the device.

FIG. 4 shows an instrument according to the invention from top view.

FIG. 5 shows different front and side views of a possible instrument set up.

FIG. 6 shows a gripper for transportation and transfer of a device on an instrument according to the invention.

FIG. 7 shows a diagram showing an instrument cycle including mixing periods and idle periods in alternating order. Furthermore, other actions in the cycle are shown.

FIG. 8 shows a diagram showing the sequential, interleaved treatment workflow of 6 devices in 6 recesses on the same treatment station some having different treatment times but the same length of the instrument cycle.

### SUMMARY OF THE INVENTION

In a first embodiment, the invention is directed to an instrument for efficient processing one or more analytical devices containing immobilized binding reagents comprising a sample input station, one or more treatment stations, a detection station, and a transfer module, wherein at least one of said treatment stations comprises a shaker unit comprising a recess for receiving said analytical device, said recess having an upper opening for placing said analytical device into said recess.

In another embodiment, the invention is directed to a diagnostic system for determining one or more analytes in a sample comprising an instrument comprising a sample input station, at least one treatment station comprising a shaker unit comprising one or more recesses for receiving devices containing immobilized binding reagents, said recess having an upper opening for placing said device into said recess, a detection station, and a transfer module, and one or more of said devices.

In another embodiment, the invention is directed to a method for determining one or more analytes in a sample using an instrument comprising the steps inserting the sample in a sample input device into an input station on said instrument, inserting one or more devices containing immobilized binding reagents into a disposable input station on said instrument, starting a controlled, automated procedure to transport the sample through a binding station and a detection station comprising
transferring the sample into one of said devices,
transferring reagents into said device,
transporting said device into said binding station,
keeping said device in said binding station under conditions allowing binding of the analyte to be determined with said binding reagents,
removing the liquid from the device,
adding and removing a washing liquid to the device and from the device,
adding and removing a stain buffer to said device,
adding and removing a washing liquid to the device and from the device,
transporting said device into said detection station, and
detecting a signal based on the binding of said analyte to said binding reagents, and
   determining the analyte based on said signal.

In another embodiment, the invention is directed to a method for automated processing of one or more devices containing immobilized binding reagents comprising the steps
providing an instrument comprising one or more treatment stations,
providing one or more devices containing immobilized binding reagents in a disposable input station on said instrument,
providing a sample in a sample input station on said instrument,
starting a controlled, automated procedure to transport said device through said instrument comprising the steps
   transferring the sample into said device,
   transferring reagents into said device,
   transporting said device into said treatment station,
   maintaining said device in said treatment station under conditions allowing said treatment,
      wherein said device is shaken during treatment in a recess of a shaker unit of said treatment station, said recess having an upper opening for placing said device into said recess.

### DETAILED DESCRIPTION OF THE INVENTION

Chips for analyzing components of a sample on their surface are well known, for example from EP 0 476 014. They are usually flat plates made from glass or other material inert to the sample and the reagents used to react the sample and its components with. One of their sides is at least partially coated by reagents that are designed to bind the components of the sample to be analyzed, if present. The area of said side which is covered by said reagents is from about 4 mm² to about 2 cm². Preferably, the surface covered is flat. The binding reagents are preferably specific for the components to be analyzed. In case of antibodies to be determined, the binding reagent may be an antigen which can be bound by the antibody. For the analysis of nucleic acids, the binding reagent may be a nucleic acid comprising a sequence which can hybridize to the nucleic acid to be determined. In case of nucleic acids, the nucleic acids immobilized to the surface are usually oligonucleotides, i.e. chemically synthesized polynucleotides. Methods for their synthesis are disclosed in EP 0 476 014. Depending upon the number of analytes to be determined in the device the corresponding number of different binding reagents are immobilized to the surface. The reagents are conveniently arranged in a geometrically fixed and defined manner. Preferably, ten or more, more preferably between hundred and one million, different binding reagents are immobilized on one chip. Those arrangements are frequently called arrays.

The chip preferably is transparent for radiation used to detect any signal created or bound to the surface of the chip pointing to the interior of the cavity.

The device according to the present invention preferably is an analytical device and preferably has a generally tubular body with a bottom wall, side walls and an upper opening which can be dosed by a cap or sealing. The binding reagents are immobilized to the body such that they are accessible to the sample, preferably on the chip as outlined above. The body preferably has a cavity with a volume of 10 μl to 800 μl, preferably 20 μl to 200 μl. This cavity is used as a process chamber to treat the liquid, e.g. a sample liquid. Thus, the cavity is designed to be at least as large as the volume of sample fluid to be treated in the device. Preferably, the volume of the chamber is at least 10% larger than the volume of the liquid to be treated. Any additional volume for reagents to be added needs to be considered. The cavity further has a form allowing the sample liquid to fully contact the binding reagents. In case of reagents immobilized on the surface of the chip, the chip is preferably located at one of the side walls of the device, such that the cavity is accessible for a pipetting device for aspirating and dispensing the sample liquid or/and any reagents without the pipetting device contacting and damaging the surface. The shape of the cavity is such that there is a distance of at least 1.5 mm from the binding reagent bearing surface to the nearest opposing wall of the cavity. Preferably, the cavity has a diameter of at least 3 mm in the region of the chip. The length of said cavity from the bottom to the upper opening is at least 5 mm, preferably between 6 and 20 mm.

In a preferred embodiment, the cavity has the shape of a cuboid having side lengths which are equal or of the same order of magnitude. That cuboid then has a side length of 3 mm or more.

In a preferred embodiment of the invention, the device as outlined above is designed to be operated in an up-right standing position. This means that one end of the tubular body is a permanently closed bottom end. The chip containing the reagents is located in the lower half of the tubular body. The other end of the tubular body contains an opening. When the device is in use, this end is preferably the upper end, and the opening will thus be designated the upper opening. The upper opening is used to introduce any liquid, e.g. the sample and reagent liquids, into the cavity. While it is possible to close the opening using a screw cap, a very preferred embodiment uses closing the opening using a pierceable membrane. Such pierceable membranes can be selected from the group consisting of silicone and polymers. Most preferred are self-sealing materials, such as elastomers, most preferred TPE (thermoplastic elastomer, melt-processable rubber). If pierced, the material should retain more than 90% or the liquid when heating for 16 hours for on 60° C. The cap is preferably manufactured by 2-component-injection-molding. A perfect fit to the processing chamber can be achieved, if the diameter of the cap in the region facing the chamber is slightly larger than the inner diameter of the chamber in this region, such that a slight pressure is maintained on the inner diameter of the chamber.

The device also comprises first 3-dimensional engagement elements (23) for accurately positioning the device, and therewith any chip held in said device, relative to device holders or other device interfaces. These first engagement elements are arranged on the device such that all degrees of freedom of the device are fixed and the position of the active surface of the carrier is defined with respect to the device during processing, detection or assembly of the device. The term 3-dimensional is intended to reflect that the elements are not solely planar surfaces or solely edges. Said elements are preferably selected from the group of grooves, recesses, projections, noses and protrusions formed from or in the surface or edge of the device. The elements are preferably made from the same material as the body of the device and are preferably integrated into to the device. The shape of the first 3-dimensional engagement element is chosen to be capable of engaging with so called first 3-dimensional constructional elements of the instrument for positioning the device.

For identification and process control in instruments the device comprises as integral part a plane and printable space (5) for labeling, e.g. with a barcode, such that the labeling process does not interfere with the carrier itself or with the fixation process of the carrier and can be carried out during or after the device assembly process. For automation and assembly purposes in a preferred embodiment the printable space is on the same face of the device as the carrier.

In a preferred embodiment, the device comprises second 3-dimensional engagement elements (22). Those second elements can be present independent from the presence of the first engagement elements outlined above. The second engagement elements are designed to pick and transport the device for instrumentation, automation or assembly purposes. In order to be accessible properly, they are preferably located at a site different from the first engagement elements, preferably on another side of the body of the device. Most preferably, they are located on the upper part of the device, preferably in pairs oppositely arranged at the sides of the device. These second engagement elements are arranged such that the carrier itself, the device assembly process and the liquid application through the device opening is not affected. Said elements are preferably selected from the group of grooves, recesses, projections, noses and protrusions formed from or in any surface or edges of the device. The term 3-dimensional is intended to reflect that the elements are not solely planar surfaces or solely edges. The means are formed at an accessible site on the device such that engagement with a second 3-dimensional constructional element of the instrument, e.g. a gripping device, designed to pick and transport said device is possible. The second 3-dimensional constructional elements are construed such that they fit in shape to the 3-dimensional engagement elements on the device. Most preferably, the engagement element is a groove in a surface of the device. Such groove preferably may be between 0.1 and 5 mm deep and preferably covers an area of between 0.01 and 0.5 cm² of the surface. The engagement elements preferably are within the upper part of the device, i.e. within the half near to the upper opening. In FIG. 1 an exemplary second 3-dimensional engagement element 22 is shown. Another first 3-dimensional engagement element is hidden on the opposite side of the device.

In FIG. 1 there is shown a device which is particularly useful in combination with the instrument according to the invention. The device (1) has a chip (2) located on the lower part of the device using a frame (4) to press the chip towards a sealing rim. A surface (5) on the upper part of the device provides space for attaching a label, e.g. a barcode. The cap (3) doses the upper opening of the device. First 3-dimensional engagement means (23) are located in the upper portion of the device. In a preferred embodiment, the device of the present invention is a device according to EP 1 419 821, which is hereby incorporated by reference to disclose the characteristics and the manufacture of said device. In addition, preferred, the upper part of the device is modified as outlined above.

The particular design of the upper opening has considerable advantages, especially useful in automated processing of samples in analytical devices containing chips with binding reagents immobilized thereon. The invention has found that exact positioning of the device within the instrument is a substantial requirement for automated procedures using chip-based assays, as exact positioning may avoid extended software procedures to attend for calculations to compensate inaccurate positioning of the device or of the surface of the chip.

A sample input station according to the invention is a part of the instrument where samples can be introduced into the instrument. Usually, the sample input station defines the position to receive the containers containing one or more sample input devices, e.g. vessels containing sample. Such vessels are commonly known. Those positions are defined such that the instrument recognizes each position as to receive a defined sample. This sample preferentially is identified by a label, such as a bar code label that can be read prior to the sample entering the instrument or thereafter or concurrently therewith. This is done by a reader located adjacently to or within the sample input station. In the present invention, the sample receiving station has at least 4 positions to receive sample vessels, preferably at least 8, more preferably between 8 and 96 sample vessels. This way, the instrument is capable of handling a corresponding high number of sample fluids without any need to stop other processes occurring within the instrument.

A sample according to the invention can be any liquid which is intended to be subjected to analysis. Usually samples are fluids, e.g. liquids, taken from the human body, like urine, sputum, blood, liquor or fluids derived therefrom, like serum or plasma. Preferred samples are fluids as above, further pretreated for better analysis. Pretreatment steps maybe chosen for the group of isolation of components, removal of components from the sample, concentration, dilution, addition of reagents, amplification of components and lysis of components. Those pretreatment steps may have been done manually, be performed on another instrument, or performed on the same instrument. The sample input station may thus also be the output station of a prior treatment station on the same instrument.

A detection station is a part of the instrument equipped with a unit for detecting a signal received from the sample upon stimulation of the sample. Means for stimulating a sample comprise irradiation by electromagnetic radiation, for instance light appropriate for exciting a component in the device which is a measure of the presence, absence or amount present of the analyte. In a preferred embodiment, the light is used to excite a label attached to a probe. The signal, i.e. the light returning from the device is then correlated with a reference signal received from a sample with known analyte(s). In a more preferred embodiment, the surface of the chip pointing to the inner of the cavity is scanned for a signal and the locations showing a signal and the intensity of the signal received from each location are identified. Those detectors may also include a confocal scanning microscopic device. Suitable scanning detectors are widely known in the art. The 3-dimensional constructional elements of the device are particularly useful to position the device in the detection station, as, for example, scanning of surfaces is sensitive to accurate positioning. The scanning process will preferably provide reliable signals from chips that are supported in a statically determinate and stress-free form.

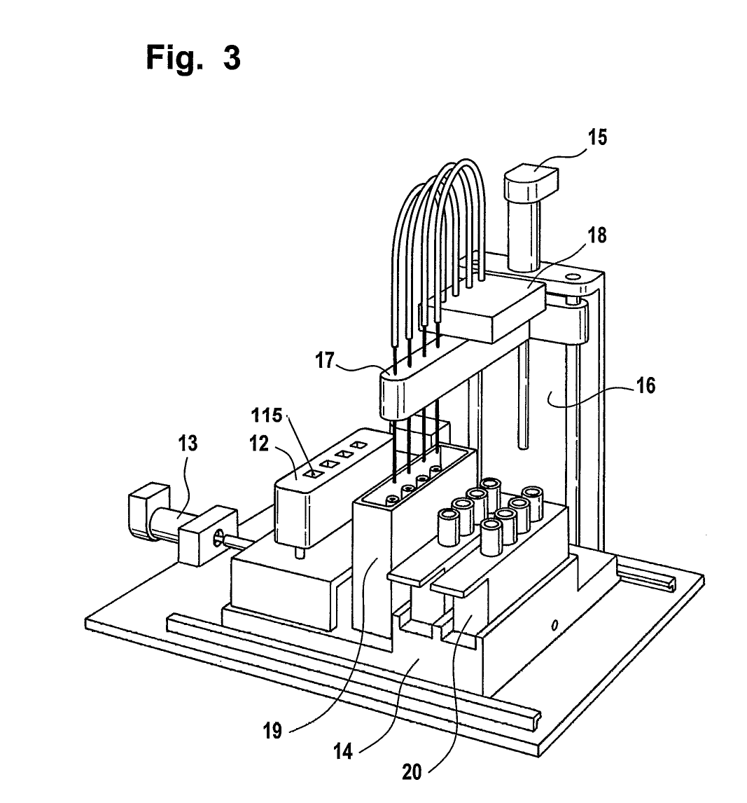

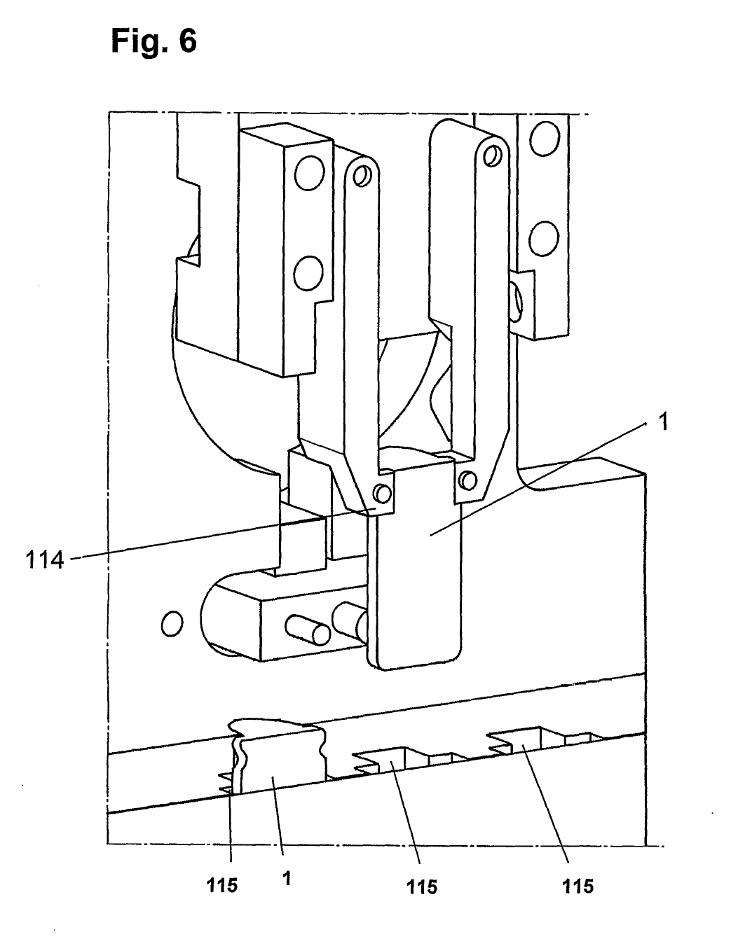

A transfer module according to the invention is a part of the instrument intended to transfer a fluid, for instance a sample fluid or a reagent or a wash buffer, or/and to transport a device, either empty or containing any fluid, from one location to another location. Thus, preferred transfer modules comprise a liquid handling unit, for example an aspiration-dispensing unit, like a socket for receiving a pipette tip or a syringe, or/and a gripper for interlocking to a device or a part of a device. Appropriate transfer modules are well known. In a first embodiment, the transfer module comprises a gripper for receiving, e.g. picking up, a device according to the invention. Preferably, the receiving is achieved by second 3-dimensional constructional elements on the transfer module fitting in form to the second 3-dimensional engagement elements of the device. The transfer module may be capable of picking up one device only or may be capable to pick up several devices at once. Preferred, the transfer is done one by one within a defined and repeated instrument cycle. This has considerable advantages in workflow, as different devices in this field may have different retention periods on the different stations, dependent upon the duration of the particular process step. An exemplary gripper is described in EP 0 907 083, and a preferred gripper for automated transport of the device is depicted in FIG. 6. In a second embodiment, there is a second transfer module for dispensing and aspirating a fluid from or/and to the analytical device. In this case, the transfer module does not need to cover the full work space, but its range of movement may be limited to a part thereof, e.g. to within the treatment station. There maybe one or more aspiration/dispensing devices, e.g. pipetting units, one for each analytical device, either operating in parallel or consecutively or intermixed. The case of parallel pipetting devices on a treatment station is shown in FIG. 3.

A disposable input station is a part of the instrument for receiving and containing unused devices, such as analytical devices as outlined above. For avoiding contamination of unused devices, the devices are stored on a station spaced apart from the treatment stations. The devices are transported by a transfer module to the treatment station for receiving liquids, e.g. sample or reagents, preferably in automated manner.

A treatment station is a part of the instrument designed for treating the device (and the liquid contained therein) during one or more steps of the analytical process. It includes a position to maintain the device containing the sample at a defined position within the station. Appropriate positioning means are first 3-dimensional constructional elements defining a holder for the device depending upon the first 3-dimensional engagement element of the device, like recesses. In a very preferred embodiment, the treatment station comprises a device carrier (12), in the following called dispo carrier, which comprises a recess having an upper opening to receive the analytical device. Preferably, each treatment station has two or more, most preferably between 4 and 48 free accessible recesses for receiving single or connected devices, most preferably single devices. The treatment station is loaded directly from top during operation at defined position marks, without opening and closing of the station. The inner form of the first 3-dimensional constructional element preferably mimics the outer form first 3-dimensional engagement element of the device, at least in the part of the device which is intended to be treated in the particular treatment station, such that the device cannot unintendedly escape the treatment station during treatment.

The intended use of treatment stations is improved application and control of typical parameters for micro array-based tests, as fluid delivery, temperature (heating and cooling) and mixing. The treatment station can have one particular purpose, e.g. providing the conditions and performing hybridization, such that any washing steps are made on a separate treatment station, but they can also be made on the same station. One particular aspect of the invention is that the concept makes it possible that more than one treatment station can be based on the same concept to handle diagnostic devices. For example, in those treatment stations, shaking is done with the same technical concept, e.g. rotational movement of the device, and liquid handling is done with the same concept, e.g. piercing from top a membrane on a device with a needle and introducing liquid into and removing liquid from said device through said needle, each being performed when said device is in up-right standing position. This has the advantage that the instrument is quite flexible such that several assays using different assay formats and having different treatment regimes and residence times in the different treatment stations can be handled on the same instrument.

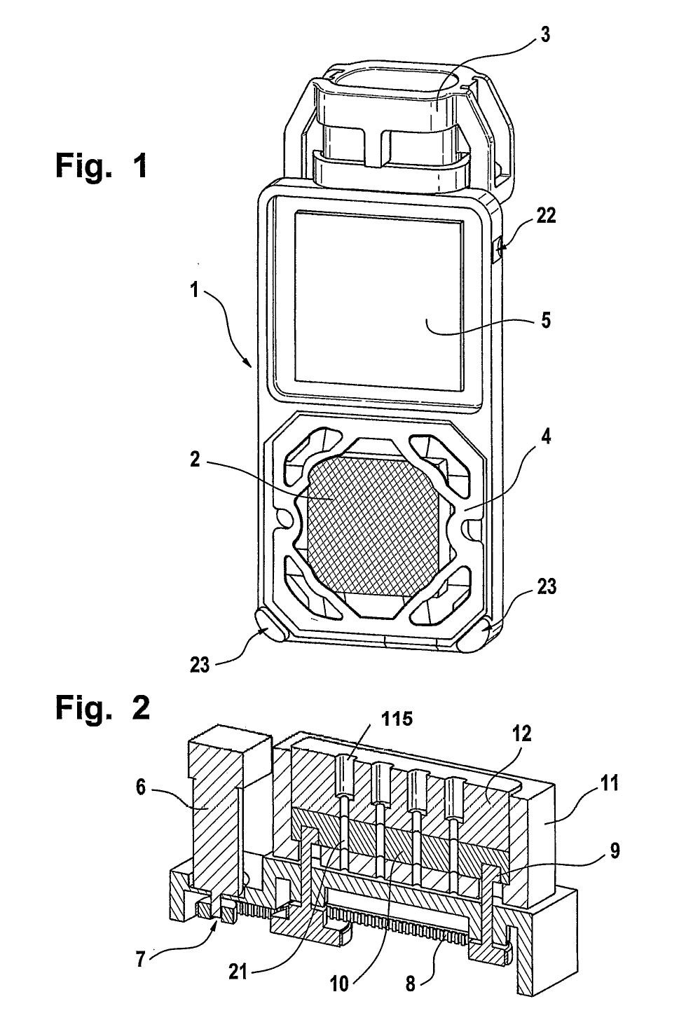

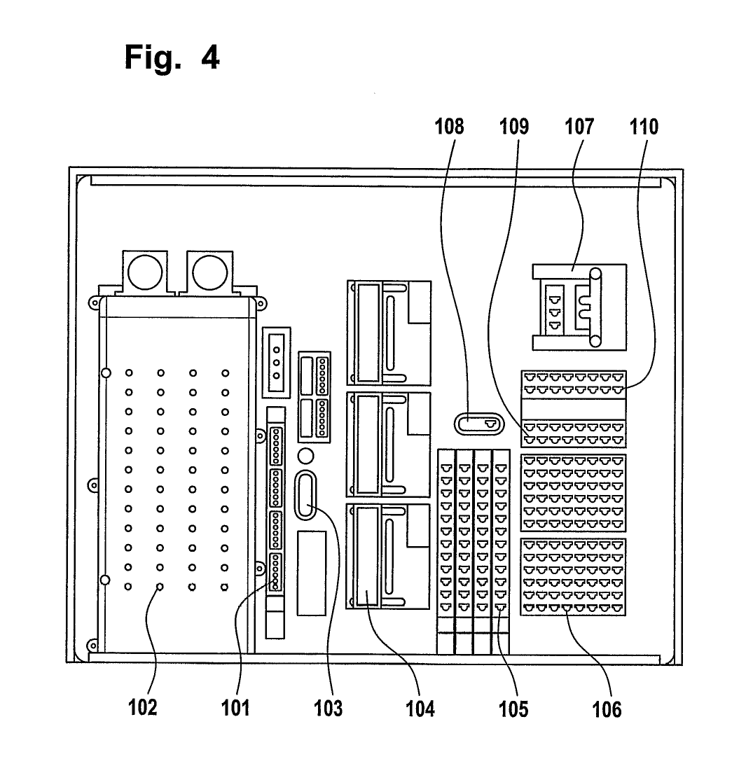

In a preferred embodiment a treatment station according to the present invention is equipped with a heating unit. Such heating unit is designed to heat the analytical device and its contents to a temperature as required by the particular treatment to be performed in the device, when contained in the treatment station. Appropriate heating units are known, for example, Peltier elements, Joule heater or resistance heating equipment. Particularly preferred, the heating unit comprises a temperature control element, for example, a temperature sensor to determine the temperature of the device or/and the liquid contained therein directly or indirectly. This may be necessary to keep the temperature of the liquid controlled within a particular range. For maintaining a desired temperature, the station may contain isolation means around the device or/and the heater. Such isolation means may be made from polystyrene or other isolating plastics and may be contained within a cover. For flexibility reasons in a preferred embodiment, see FIG. 4, a treatment station can also be set up with different segments (109, 110) applying different temperatures where every segment has an isolation. In order to enable temporary temperature changes or the use of temperature profiles the treatment station may comprise one or more cooling ducts (see for example (21) in FIG. 2). Cooling may be effected by known means, but preferably the duct allows for external ventilation of the devices and the desired temperature control of the device.

In another embodiment, the treatment station is equipped with a cooling element. Cooling elements again are generally known. Preferably, the cooling element comprises vents in the treatment station to provide a stream of air of a temperature lower than the temperature to which the cooling should result. The cooling process may be enhanced by providing mechanical ventilation devices, such as a ventilator.

In further another preferred embodiment, the treatment station may comprise a needle cleaner. Such device is advantageously located in the near neighbourhood of the recesses of the same treatment station, e.g. not farther away than 10 cm. A needle cleaner is a device allowing removal of remnants from the contents of the device after removal of the needle from the device. Those remnants may disturb the treatment performed in a device on the same treatment station which uses the same needle for liquid handling. Needle cleaners are commonly known to those skilled in the art and are preferably based on introducing the needle into a wash solution and then removing the wash solution. The interior of the needle may be cleaned by sucking the wash solution into the interior of the needle and spitting it into a waste container. Those cleaning processes may be performed repeatedly as required.

As essential feature of the present invention is that the treatment station comprises a shaker unit. The shaking of the device improves typical micro array-based treatments as binding, staining and washing. A shaker unit is an equipment which is used to shake the device containing the sample. Shaking is a process of moving the device such that compartments of its content get mixed. So for instance application of ultrasound to the device is not considered shaking, as it does not move the device. Furthermore, vortexing is also not considered shaking, as it does not move the device. Preferably, the shaker is driven by a mechanical drive, for example a motor. The preferred motion comprises at least movement in a plain (X-Y-axes). However, the movement can also have a component in a direction perpendicular thereto (Z-axis). More preferably, the X-Y plain is substantially perpendicular to the earth gravity force. Furthermore, the X-Y plain is substantially perpendicular to the surface to which the binding reagents are coated.

There are several modes of realizing such shaking. In FIGS. 2 and 3 there is shown an embodiment using an eccentric movement of a carrier for disposables (in the following called disposable carrier) containing a number of devices, that uses a simple continuous rotational movement instead of back and forth movements which requires more complicated mechanisms. A drive (13) has a rotational axis which feeds a drive belt (8) which in turn is used to rotate a first axis connected to the disposable carrier, e.g. by an eccentric (9), such that rotation of the axis yields in eccentric, cyclic movement of the carrier. A second axis is connected to the first axis to move with the same frequency and amplitude, which gives a circular movement of the complete disposable carrier. The devices can be inserted into said dispo carrier in recesses (e.g. shown as (115) in FIG. 2.

Preferred treatment stations are selected from the group consisting of binding stations, staining stations and washing stations, or any combinations thereof, like a combined binding and staining, a combined staining and washing, a combined binding and washing and a combined binding, staining and washing station. The design of said station is determined by its function. Thus, the functions of the treatment station are selected from the function of binding, staining and washing. Those treatment stations may require some, but not all measures as pointed out above. For example, a washing station may use fluid handling (e.g. aspiration and dispensing), mixing and optionally heating or/and cooling, a binding station may need mixing and heating or/and cooling and a staining station may require mixing and heating or/and cooling.

In a first preferred embodiment, the treatment station is a binding station. A binding station preferably provides all conditions needed for efficient binding of components of the sample to one or more of the binding reagents immobilized in the device. Efficient binding preferably is achieved by delivering sample and binding reagents into the device, and keeping the fluid within the device at a defined temperature. Preferred temperatures for binding nucleic acids to capture probes are between 20 and 95° C., more preferably between 40 and 60° C. For reaching and controlling an intended temperature, the binding station preferably has a heating element, a cooling element and is equipped with a shaker for improved binding and shorter reaction times. Introducing and removing devices and delivering fluids ask for defined and precise shaker positioning.

In a preferred embodiment, which is shown on FIG. 2, the binding station contains a dispo carrier (12) made from heat conductive material, preferably metal, most preferably aluminum containing the recesses for four devices, an isolation (11) surrounding said dispo carrier, a heater (10) made from metal or ceramics heating elements, cooling ducts (21) within the carrier and heater and a shaker drive (6) connected with the carrier (12) by an eccentric (9).

In a second embodiment, the treatment station is a staining station. Staining is a process to visualize any components bound to the binding reagents immobilized to the device. It is mainly used in case the components are not directly detectable, but need further reagents to develop a signal. Such reagents may be compounds being capable of binding to the components bound to the device. In an exemplary assay, the components of the sample to be analyzed are nucleic acids labeled with biotin. In this case, staining can be done by delivering sample and a conjugate of avidin or streptavidin and a fluorescent label into the device, and keeping the fluid within the device at a defined temperature. Preferred temperatures are between 20 and 60° C., more preferably between 20 and 40° C. After completion of the binding reaction of biotin to (strept)avidin the resulting complex will have fluorescent characteristics. For reaching and controlling an intended temperature, the staining station preferably has a heating element, a cooling element and is equipped with a shaker for improved staining and shorter reaction times. Introducing and removing devices and delivering fluids ask for defined and precise shaker positioning.

In a third embodiment, the treatment station is a washing station, as depicted in FIG. 3. Washing is a process to remove unwanted components of the sample from the bound components. To achieve this, after completion of the binding reaction the liquid is removed from the device, while any components bound by the binding reagents will remain in the device. A washing liquid is added to the device to further dilute any remaining undesired components which may still adhere to the device. The washing liquid is removed from the device together with the undesired components. This process preferably is repeated as often as necessary to remove undesired components to a concentration not interfering with the determination of the intended analyte. In order to improve and control the washing and dilution process and to reduce the necessary wash cycles the washing station is equipped with a shaker. Introducing and removing devices and delivering fluids ask for defined and precise shaker positioning. The washing liquid has a chemical constitution which does not substantially affect the binding of the analyte to be determined. For efficient automated processing a multiple needle module (17) is mounted to a vertical carriage (16) moved with the drive (15) and the device carrier (12) is mounted to a horizontal carriage (14) moved with the drive (13). In order to eliminate any possible contamination effects between different probes or between different assay steps or between any other liquids, and in order to optimize wash dilution the needle module (17) can be cleaned in each step with a needle cleaning module (19), which is a significant improvement compared to micro array processes based on fluid exchange with adapters, i.e. pumps or docking stations. For reaching and controlling an intended temperature, the washing station preferably has a heating element and a cooling element.

One advantage of the present invention is that it provides reliable and efficient binding, staining or washing in devices containing immobilized binding reagents. Particularly, it is possible to realize those advantages for more than one device without substantial differences in parallel analyses. It also allows for precise and prescribed movements and positioning of the disposable carrier which is of advantage for automation of device handling and processing.

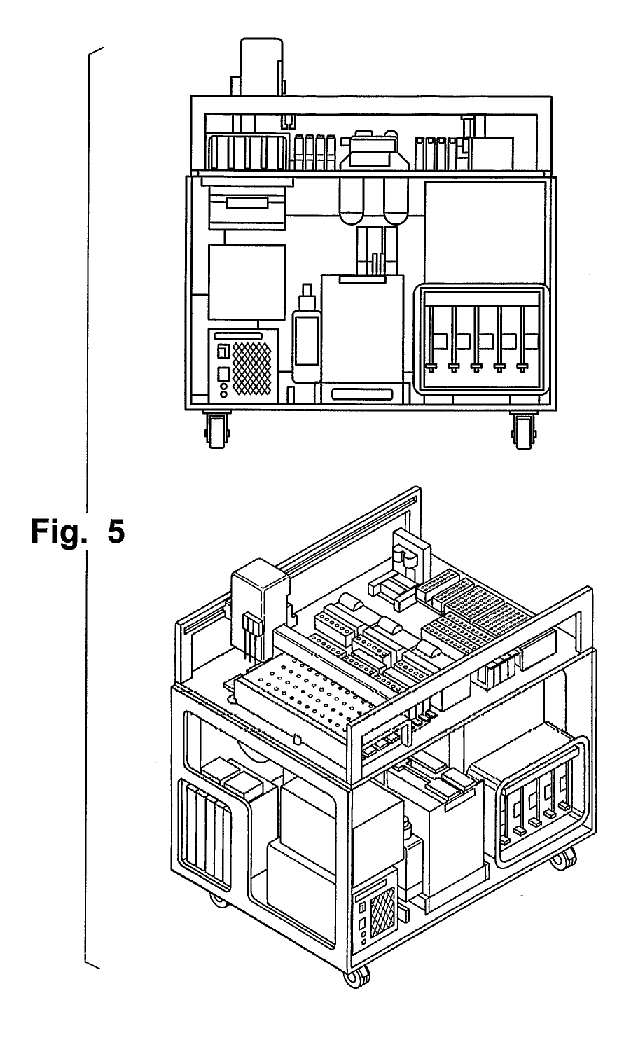

An exemplary instrument according to the invention is shown in FIG. 4 and FIG. 5.

Another subject of the invention is a method for determining one or more analytes in a sample comprising the steps
inserting the sample in a sample input device into an input station on said instrument,
inserting one or more devices containing immobilized binding reagents into a disposable input station on said instrument,
starting a controlled, automated procedure to transport the sample through a binding station and a detection station comprising
   transferring the sample into one of said devices,
   transferring reagents into said device,
   transporting said device into said binding station,
   keeping said device in said binding station under conditions allowing binding of the analyte to be determined with said binding reagents,
   removing the liquid from the device,
   adding and removing a washing liquid to the device and from the device,
   transporting said device into said detection station, and
   detecting a signal based on the binding of said analyte to said binding reagents, and
   determining the analyte based on said signal.

Another embodiment of the invention is a method for determining one or more analytes in a sample using an instrument or a system as defined above.

Methods for determining an analyte in a sample based on chip technology including an array of reagents, e.g. probes, immobilized on a surface are generally known to a man skilled in the art. A wide range of chips are commercially available from companies including Affymetrix Corporation. In general, the most convenient methods comprise pre-treatment of analytes contained in a sample to amplify specifically or unspecifically sequences contained in a nucleic acid to be determined. Such amplification is conveniently done using the polymerase chain reaction. The choice of the sequences of the primers will determine which analyte sequences will be amplified and can be determined later on. Other methods include in vitro or in vivo expression of particular nucleic acid sequences from the sample. Such methods are also well known.

Furthermore, pre-treatment of a sample according to the present invention includes labelling the analyte to be determined, e.g. the nucleic acid to be determined, with a label capable of providing a signal that can be detected in the instrument according to the present invention. Appropriate labels and methods for attaching the label to the analyte are well known to those skilled in the art.

In an initial step, according to the method of the present invention, the sample containing the analyte or the analytes or any compounds derived therefrom in the pre-treatment step, like amplificates or expression products derived from the analyte are inserted into an input station on the instrument on which subsequent steps will be performed. Conveniently, the sample is contained in a sample input device, preferably in a tube, which is the output device from the pre-treatment. The insertion can be performed manually or by an instrument, e.g. by a robot arm on an instrument. If one or more of the pre-treatment steps is performed on the instrument used according to the present invention, the transfer module used for later steps can conveniently be used in the pre-treatment steps or for the insertion of a sample input device.

The analytical devices to be used in the method of the present invention are provided on a disposable input station on the same instrument as the sample. Devices can be inserted into the instrument either prior to, concomitantly with or later than providing the sample input devices on the instrument. However, the sample input devices and the analytical devices shall be available on the instrument prior to starting the following automated procedure. The analytical devices are conveniently provided on one or more so called racks, each containing an appropriate number, e.g. between 6 and 50, preferably between 8 and 30 devices. Those devices can be different or can have identical reagents immobilized. Preferably, in the present invention there are at least two different devices provided on the disposable input station. Those devices differ in the sequence of the binding reagents immobilized on the chip contained in the devices. For example, one of the devices may contain probes having sequences for the determination of expression profiles and another device may contain probes for the detection of amplicons prepared by the nucleic acid amplification reaction.

A disposable input station according to the invention is a station, where one or more disposables useful for the method according to the invention are stored ready for use in the method. This disposable may be selected from the group of pipette tips, vessels and the analytical devices containing the immobilized binding reagents. The insertion can be made both manually or automatically.

The core feature of the method according to the invention is the performance of a controlled automated procedure including the transport of a sample through a binding station and a detection station. This automated procedure may include further steps of transport of the sample or products derived therefrom, preferably in the analytical device as mentioned above, within the instrument. As will be explained later, the automated procedure is controlled, such that for a particular method the required steps are performed in an automated manner, substantially without manual intervention.

A first series of steps is performed to achieve a status that the sample and any reagents necessary for binding the analyte or any compounds derived therefrom are brought into contact with the reagents immobilized in the analytical device. The order of steps to achieve this result is essentially unimportant. Preferably, the analytical device is inserted into the binding station, then the sample is introduced into the cavity of the device, and then the reagents are added to the sample in the device. Preferably, transporting of the device into the binding station is made using the transfer module of the instrument according to the invention. Preferably, the transfer module comprises three dimensional constructional elements for gripping the device through three dimension engagement elements of the device. For details of those elements, reference is made to the description of the instrument according to the invention. In detail, the preferred mode of this step comprises gripping the device by the transfer module, raising it vertically (along the Z-axis of the instrument) moving the transfer module horizontally (in X-, Y- or X-Y-direction) over the working area of the instrument and then lowering the device (in Z-direction, vertically) through the upper opening of the recess of the binding station. Then, the transfer module releases the device such that it remains in the binding station. The transfer module conveniently will be moved to a different place on the instrument. If more than one analytical device is used in the method, the transfer module may transport another device into another recess contained on the binding station.

Transferring a sample or reagents into the device can be performed by a second transfer module or preferably is done by the same transfer module. The transfer module for transferring a sample or reagents is preferably equipped with a liquid transfer device. A liquid device useful for the present invention is a means capable to aspirate and dispense liquids, like sample, reagents, washing liquids or reaction mixtures, into and from the analytical device, respectively. Suitable liquid transfer devices are known in the art and include reagent pipetting needles and pipette tips. In the preferred embodiment of the present invention, where the analytical device contains a pierceable opening, the liquid transfer device must be capable of piercing the pierceable membrane closing the opening. Therefore, steel needles are highly preferred in this embodiment. A particular advantageous embodiment of the present invention is when filling the device and washing it using the same liquid handling principle, e.g. by introducing a needle through a pierceable membrane of an up-right standing device.

The step of transferring the sample into the device will preferably comprise moving the transfer module, preferably comprising an aspiration dispensing unit vertically to a place on the instrument, where the sample is positioned. This may be the sample input station or a station, where any pre-treatment steps, like labelling of the analyte, has been performed. The sample will aspirated into the transfer module, the sample be moved to a position above the upper opening of the analytical device, then be vertically moved such that the liquid transfer device is introduced into the analytical device positioned on the binding station. This may include piercing of a pierceable membrane, when moving downwards.

Similarly, reagents needed for the binding process may be aspirated and dispensed using the liquid transfer device. For this process, the same or different liquid transfer devices can be used. However, in case reagents are to be transferred after a transfer of a sample, the liquid transfer device should either be changed, as in the case of a disposable pipette tip, or extensively cleaned in case of a useable liquid transfer device, e.g. a steel needle. Cleaning modules are generally known and can be positioned either on the binding station or on a different part of the instrument, which can be accessed by the transfer module used for the liquid transfer.

During the transfer and transporting steps, the binding station is not subjected to a shaking process. This can be achieved by programming the control in accordance with a defined and repeated instrument cycle. This instrument cycle is preferably clocked. The term clocked in the present invention means that the overall treatment time in a particular treatment station, and more preferably on all treatment stations in the instrument, can be divided in constant intervals of substantially the same length beginning and ending at the same time at the various treatment stations. Those instrument cycles are programmed to allow certain actions at certain predefined times in the interval. This means that while the instrument cycle length may be identical for a given treatment station, the actions performed in the instrument cycles may differ, dependent on the requirements of the assay, particularly on the assay format.

For binding the analyte to be determined to the reagents immobilized in the device, the device is kept in the binding station under conditions allowing binding of the analyte to be determined to the binding reagents. Extremely preferred in the method according to the present invention, this step contains at least one period of shaking the device in the binding station. For this purpose, the binding station is adapted to be shaken and therefore acts as a shaker unit.

The shaking process according to the present invention can be performed in several ways. Preferably, a shaker unit is equipped to the mechanical drive allowing for shaking with the frequency of between 10 and 50 Hertz. In terms of the amplitude used for shaking, a mechanical drive preferably allows shaking with an amplitude of between 0.1 and 10 mm. Preferably, the shaker unit comprises a drive unit to lead that device on a predetermined path. Said path may be a circular path or an elliptical path.

The shaking process is preferably controlled by a computer program. The computer program preferably induces intervals of shaking periods and idle periods in the shaker unit. More preferable, the intervals of shaking periods and idle periods are regular. Those intervals may be between 1 and 1000 seconds long. Preferably, the intervals are between 1 and 60 seconds, most preferable between 5 and 20 seconds long. Shaking periods preferably are shorter than idle periods. Preferred length of a shaking period is between 1 and 200 seconds, more preferable between 2 and 60 seconds and most preferable between 3 and 20 seconds. Length of idle periods is preferably between 2 and 200 seconds, more preferable between 5 and 60 seconds and most preferable between 10 and 30 seconds. Obviously, the shaking period is followed by an idle period, which in turn is followed by a shaking period. Preferably, between 2 and 20, more preferable 3 and 10 and most preferable 4 and 8 intervals, each containing a shaking period and an idle period, are performed. Shaking and idle periods are embedded in the overall instrument cycle.

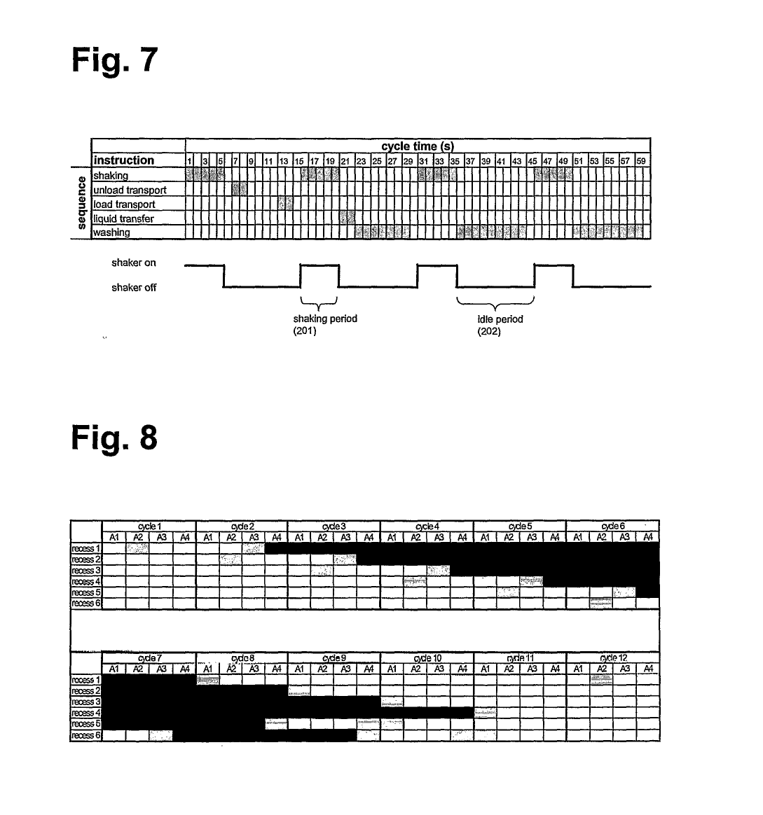

FIG. 7, upper part, shows an exemplary series of intervals of shaking periods (201) and idle periods (202). During the times when shaking is turned off (i.e. during idle periods), any transfer actions, e.g. transferring any samples into a device positioned in the recess of the binding station or transport action of device into a treatment station or removing liquid from the device or removing a device from the treatment station, can be performed. Importantly, the shaker unit is controlled such that in the idle periods, the analytical device is located a predefined position. This allows for free access of the transfer module to the analytical device in the treatment station without conflict. This requires exact positioning of the device in the shaker unit of each treatment unit. This is preferably facilitated by the first dimensional engagement elements of the device and/or the first dimensional constructional elements of the shaker unit. Most preferable, there is a defined position in the shaker drive, where all analytical devices in one treatment station are located such that they are accessible from top by the transfer module, preferably just below the gripper or the aspiration/dispensing device, so that approaching the transfer module to the device merely requires a vertical movement (Z-axis).

After sufficient incubation of the sample with the reagents immobilized in the device in the binding station, the liquid is removed from the device. The analyte bound to the immobilized binding reagents remains in the device during this step. In order to perform such removal of the liquid from top, the device is either transported from the binding station to another position on the instrument or is kept in the recess of the binding station. The removal of the liquid is preferably done using a transfer module, more preferable the transfer module also used for transferring the sample and the reagents. So the same general instructions apply for the removal of the liquid. Particularly, a liquid transfer device is inserted through the upper opening of the device into the cavity of the device, the liquid is aspirated, the liquid transfer device is moved upwards, moved to a position where discarding of the liquid can be performed, for example, into a waste container, followed by washing of the liquid transfer device, if intended to be reused.

After removal of the liquid, a washing liquid is added into the device. Such washing liquids are generally known. They are chosen such that the liquid does not impair the binding of the analyte with the binding reagents, but dissolves all components not intended to be bound by the binding reagents, for instance, nucleic acids having a sequence not intended to be bound by probes immobilized on a chip.

The components not intended to be bound are removed together with the washing liquid from the device by an aspiration dispensing step, as explained above for the first liquid. There can be as many washing steps as required for sufficient removal of unwanted constituents in the device.

In an optional step, the device and the components immobilized therein are treated with the reagents for staining the analyte bound. This can be done in the treatment station as where the binding has occurred or can be performed in a separate treatment station. The station wherein the staining reaction is performed is called the staining station, irrespective of whether functions such as binding or washing are additionally performed therein. Generally, staining of analytes immobilized on a chip is well known. Usually, staining reagents, dissolved in a buffer, are added to the bound analyte. A stain can be any chemical or enzymatic compound, which can be detected or made detectable in the subsequent detection step. Appropriate stains are for example fluorescent compounds or compounds capable of binding to the analyte labelled by a compound detectable or capable to be made detectable. The liquid containing the staining reagents, in the following called “stain buffer” is designed to substantially not impair the binding of the analyte to the binding reagents.

Analogously to the addition and removal of a washing liquid, the stain buffer is added to the device and removed from the device using the transfer module.

In order to completely remove superfluous staining buffer that could simulate signal not caused by the presence of the analyte, the device is subjected to a washing procedure as outlined above.

The staining procedure is preferably done in a treatment station separate from the binding station. It has been found that separating the station for performing the binding, particularly hybridization step from the station for performing the staining procedure, can remarkably increase throughput of samples on the instrument. This is particularly true, if more than one analyte, particularly if determined by different assay formats, are performed on the same instrument.

Furthermore, the invention has found that in order to achieve such throughput in a convenient manner, it is preferred to have an automated procedure, which is controlled by a computer program. Even more preferably, the computer program is characterized to induce regular intervals of shaking periods and idle periods in the shaker units. Those intervals are most preferably in register for the different stations involved. In register for the present invention means that instrument cycles at the same and at different treatment stations start at the same point in time. For example, using regular intervals facilitates transfer of liquid and analytical devices from one station to another station by the transfer module. The intervals are kept relatively short to allow flexible transfer and transport during idle periods. Therefore, preferably the idle periods of the station from which a transfer or transport is to be made, and the station to which the transfer and transport is intended, have an overlap in time. Furthermore preferred, the shaking periods have identical length, as do the idle periods, while the shaking periods are shorter than the idle periods. The shaking of the device has the effect that the content of the device is mixed. Thus, the term shaking is in effect the same as the term mixing. In addition, preferably, there are periods, in the following called waiting periods, during which there is no mixing/shaking and no other action on the same device. These periods may have the reason that the transfer module just performs other actions and is not ready for acting on the actual device, or that a change of modules is performed, for example when the gripper moves away from the device and the aspirating/dispensing device approaches the device. But the waiting periods may also just serve the purpose to allow incubation of a liquid in the device with reagents in the device or with the device.

Or the waiting period may be needed because the particular action as scheduled in the instrument cycle is not needed for the particular device in the particular cycle.

In FIG. 7, a scheme of actions selected from the group of shaking (by the shaker unit), device transport (by a gripper on a transfer module), fluid transfer (by a pipette of a transfer module) and washing (sip and spit by a needle of a transfer module) is shown. One instrument cycle having a duration of 60 seconds is shown. The scheme shows the actions on one selected recess in one selected treatment station, here a washing station. Actions performed on this washing station are placing a device in the recess, introducing a washing liquid, shaking the device to enable efficient washing, removing the washing liquid containing any contaminants and removing the device.

In the lower portion of FIG. 7, there is shown a diagram giving exemplary shaking periods and idle periods. During the ‘on’-periods (201), mixing is done in the treatment station by the shaker unit, and during ‘off’-periods (202), the device stays at a defined position in the treatment station without shaking. As can be seen, the shaking periods each have the same length and the idle periods have the same length, which is different from the length of the shaking periods.

In the upper portion of FIG. 7 there is shown which action is performed at which time in the instrument cycle (in seconds). Grey areas indicate action of the particular module. For example, the exemplary cycle starts with 5 seconds mixing (shaking period). Thereafter, no particular action selected from the above actions is performed. Then, an idle period follows in which during 2 seconds a device can be removed from the incubator, i.e. the incubator is unloaded and the device transported to another location, e.g. to another treatment station. During the same idle period the treatment station can be loaded or re-loaded with a new device, in the example shown in FIG. 7, after 4 seconds a new device can be introduced into the treatment station at the particular recess. From second 21 onwards, for two seconds, washing liquid can be transferred into the chamber of the device. During the next 3 idle periods a device can be washed in the incubator by the washing unit (sipping and spitting the washing liquid completely or partially into and out of the chamber of the device using a needle connected to a syringe. At the end of the dedicated instrument cycle, another instrument cycle will start in the same recess (until the tasks to be performed on the instrument are completed). In this following cycle, any or all of the process steps performed in the first cycle can be performed, in accordance with the requirements of the particular treatment and device, dependent upon the overall process to be performed in the device. In the above example of a washing station, it may be required to repeat the washing process in the same device with fresh (unused) washing liquid. Thus, in the second cycle, the steps ‘unload transport’ and ‘load transport’ will simply be omitted. During the periods reserved for these actions, there will be no particular actions. All other actions during this cycle will be performed as in the earlier cycle, i.e. at second 21, the fresh washing liquid will be added, during the idle periods there will be active washing by sip and spit and during the shaking periods, the shaking will be performed. After performing as many cycles as required for removing the contaminants the cycle before the last cycle will comprise transferring the last washing liquid from the device into a waste container. Obviously, no further sip and spit will be needed during this cycle. The last cycle will then comprise in second 7 the transport of the device into the next treatment station, e.g. the detection station. In the next cycle, the recess can be loaded with another device starting second 13. Depending upon the task, the number of cycles to be performed may then differ from the number of cycled for the device before. In the example, the number of cycles used for sipping and spitting may be increased or diminished compared to the earlier treated device.

Further to the process shown in FIG. 7 another preferred mode of operation is possible in that the device(s) remain in the same treatment station during filling, hybridization and washing. Obviously, this will need special sequences for the idle periods of the treatment station that allow in addition for the actions needed for hybridization. Such process will preferably in one of the idle periods of a first cycle comprise the transfer of the sample liquid into the device and in a later cycle comprise the transfer of the sample liquid (which maybe deprived of components bound to the device) from the device. This can conveniently be done during the periods called ‘liquid transfer’ (and designed for washing liquid transfer) in FIG. 7. However, it is possible to provide extra periods of sample liquid transfer for such sample transfer. After completion of the hybridization procedure, the washing procedure can be made in the same or a different recess using the steps as described above.

The same combination of steps can be made for the staining station, i.e. combine reacting the surface in the device with reagents for staining and washing the device from superfluous staining reagent.

A cycle describes the time necessary and a sequence of operations between identical instrument actions. The cycle considers all possible actions necessary and allows a clocked processing of all assay formats running on an instrument. The cycle is repeated on the instrument for different devices and for each diagnostic test respectively. A cycle is preferably between 5 sec and 5 min long, but is identical for all devices. If different tests with different treatment times are running simultaneously on the instrument the actions performed for each device in each cycle may be different, though. I.e., not all possible actions are performed for each device. The cycle length is given by the process times for device treatments as hybridization, washing and detection, where the processes can last for several cycles. If so, the treatment stations require several recess positions.

After removal of the staining buffer, the device may be transported into a detection station. The detection station is designed to allow detection of the analytes bound to the immobilized binding reagents using a signal directly or indirectly created by the use of the reagents in the staining buffer. Preferably, the detection station comprises a scanner for detecting the signal based on the binding of the analyte or the analytes to the binding reagents. Particularly, in this station, it is important that in a preferred embodiment of the invention, three dimensional engagement elements, preferably the first three dimensional engagement elements, interact with three dimensional construction elements in the detection station to exactly position the analytical device within the detection station. This positioning is both vertical and horizontal. Transport of the analytical device into the detection station can again be made by the transfer module, particularly by the gripper with three dimensional construction elements for gripping the device through the second three dimensional engagement elements. While it may be possible in the future to scan more than one chip in the analytical device in parallel, it is a preferred embodiment of the present invention to submit only one analytical device at a time to the signal detection. Therefore, it is highly preferred that the analytical devices are not connected to each other, but are essentially single devices that are transported to the detection station one at a time, preferably in register with the shaking and idle periods in the treatment stations. The use of single device processing also has the advantage that the device including the array chip provides for direct and traceable sample-result correlation.

The step of determining the analyte based on the signal received from the device can be performed according to procedures as perfectly known to the man skilled in the art.

In a last step, the analytical device may be discarded following transfer from the detection station to a waste station. Again, this can be done using the transfer module of the instrument according to the invention.

The automated process is preferably controlled by a computer. On the computer a program is loaded that initializes the starts and terminations of the various steps performed on the stations. For example, the computer program initiates the start of the shaking procedure and terminates the shaking procedure at the end of the predetermined and stored shaking period. Furthermore, the program initiates the various movements of the transfer module in X-, Y- and Z-direction. In addition, the computer program controls the aspiration and dispensing step for the transfer unit. Such computer program will preferably contain different predefined procedures for each analyte to be determined and each format to be used. The program will require input from the operator regarding the analyte to be determined and, if several assay formats are available, the choice of the assay format. Such information from the operator may be entered through a keyboard or a sensitive monitor or by information provided on the analytical device or the sample input device. The instrument will thus preferably be equipped with units for reading information contained on the outer surface of the sample input device or/and the analytical device. This can be made by commercially available bar code labels and readers.

In a more complete and integrated procedure, the method according to the present invention comprises the following steps:
1. load the sample, the reagents and the diagnostic devices
2. run the sample preparation (e.g. cleavage)
3. if time delay cool down and hold samples
4. transfer the sample to the diagnostic device
5. hybridization
6. wash
7. staining
8. wash
9. transfer to a detection station
10. transfer to a waste station

One advantage of the present invention is that by the provision of the shaker unit in the treatment station it provides reliable and efficient binding, staining or washing in devices containing immobilized binding reagents. Another advantage of certain embodiments of the invention is that the overall process, particularly in case of different assay formats on one instrument, is quicker than in the prior art.

Another subject of the invention is a method for automated processing of one or more analytical devices containing immobilized binding reagents comprising the steps
providing an instrument comprising one or more treatment stations,
providing one or more analytical devices containing immobilized binding reagents in a disposable input station on said instrument,
providing a sample in a sample input station on said instrument,
starting a controlled, automated procedure to transport the analytical device through said instrument comprising the steps
   transferring the sample into said device,
   transferring reagents into said device,
   transporting said device into said treatment station,
   maintaining said device in said treatment station under conditions allowing said treatment
      wherein said device is shaken during treatment in a recess of a shaker unit of said treatment station, said recess having an upper opening for placing said analytical device into said recess.

The various steps of this process have been outlined in the above description. Preferably, and referring to FIG. 4, the method for automated processing of one or more analytical devices containing immobilized binding reagents according to the invention preferably contains the following steps:

In a first step, the samples are loaded into the sample loading area (101). FIG. 4 shows a rack of 24 sample vessels, in 4 discrete plates, each having 6 recesses to keep a sample in each recess. For this, the incubator (104) is opened, the incubator is loaded with sample tubes. Reagents are aspirated from the reagent bottles contained in reagent storage (102). The sample tubes are opened, if required. The reagent is pipetted and dispensed into each of the sample tubes. The sample tubes are closed, if dispensing was effected following opening the tube. The needles are washed. Then the sample tube is incubated at 40° and up to 95° C. During this step the amplified nucleic acids are transformed (cleaved) into shorter pieces. In the staining step, the following steps are performed: The incubator is opened, reagent is aspirated, the sample tubes are opened, and the reagent is dispensed into the sample tubes, or, in case of a tube closed by a pierceable cap, reagent is added by the needle reaching into the tube through the pierced cap. The sample tubes are closed, if opened before, the needle is removed from the tube, and the needle is washed. The mixture is incubated at 40° and up to 75° C.

For binding, the following steps are performed: get sample and hybridization buffer, fill chip disposable through pierceable cap, pick and place device into hybridization station (106), heat and mix, at 60° C. up to 16 h.

Washing is made as follows: pick and place device into wash station (107), wash with washing buffer A (multiple times) through pierceable cap, mix during wash procedure, wash needle each time, fill device with stain buffer.

For staining the following steps are performed: pick and place device into stain station (109), mix during staining,

Another washing is performed as follows: pick and place device into wash station (107), wash with washing buffer B (multiple times) through pierceable cap, mix during wash procedure, wash needle each time, fill device again with stain buffer.

The detection is performed by pick and place device into scanner inlet (108) and start detection.

A liquid transfer device for entering the sample to the device may be a part of the instrument which is used to supply the sample to the diagnostic device of the invention. This is preferably done automatically. Convenient means are pipetting means that can be controlled by a computer. Such pipetting devices are generally known and can be used in the present invention. Conveniently, the device comprises a socket for receiving a pipette tip and a pump for applying a slight vacuum to the interior of the pipette tip, such that, if the lower opening of the pipette tip is in contact with the sample, sample is sucked into the pipette tip. After aspirating the sample, the device is moved to the device according to the present invention, inserting the tip of the pipette tip through the inlet port into the device according to the invention. Then, the liquid is released and dispensed into the device. The same is done for any reagents needed for the reaction.

In FIG. 5 there is shown an instrument according to the invention having all elements for convenient analysis of a sample. While the modules on the working surface as shown in FIG. 4 have been described above, there are other components shown in FIG. 5. A transfer unit (111) is located above the work surface and can reach the stations as required and described above. A waste unit (112) is located below the work surface to receive fluid and solid waste, e.g. used disposables, like analytical devices, from the work surface. A computer (113) is also located below the work surface to control the process.

In FIG. 6 there are shown details of a transfer module according to the invention, in use with an analytical device (1). A gripper engages via 3-dimensional constructional elements (114) with the device and carries it from one station to another station. Also shown are three recesses (115) in the treatment station, one already being occupied by a device.

Yet another subject of the invention is a method for performing an analysis in a device using an instrument comprising treating said device in said instrument during at least two shaking periods and at least two idle periods by performing at least two actions selected from the group of
loading said device into a recess on a treatment station on said instrument,
transferring a liquid into said device when located in a recess on a treatment station on said instrument,
unloading said device from a recess on a treatment station on said instrument, and
washing said device in a recess on a treatment station on said instrument,
   wherein said actions are performed on the device in the same recess at fixed and non-overlapping action periods within repetitive instrument cycles of substantially the same length, said action periods not overlapping with said shaking periods.

The definitions given above apply to this aspect of the invention, too. Shaking periods are periods of time during which the device in the particular recess is subjected to shaking. The shaking process provides mixing of the liquid contained in the device. An idle period is a period of time during which the device in the particular recess is not subjected to shaking. During idle periods, actions as discussed herein can be performed, e.g. loading said device into a recess on a treatment station on said instrument (e.g. by a gripper on a transfer module), transferring a liquid into said device when located in a recess on a treatment station on said instrument (e.g. by a pipette of a transfer module), unloading said device from a recess on a treatment station on said instrument (e.g. by a gripper on a transfer module), and washing said device in a recess on a treatment station on said instrument (e.g. by sip and spit by a needle of a transfer module). During idle periods, the device will be positioned at a predefined position within the instrument and the treatment station, such that it can be accessed by devices used for treating said device, e.g. a gripper or a pipette tip.

Preferred, the shaking periods have identical length, as do the idle periods, while the shaking periods preferably are shorter than the idle periods. The shaking of the device has the effect that the content of the device is mixed. Thus, the term shaking is in effect the same as the term mixing. In addition, preferably, there are periods, in the following called waiting periods, during which there is no mixing/shaking and no other action on the same device. These periods may have the reason that the transfer module just performs other actions and is not ready for acting on the actual device, or that a change of modules is performed, for example when the gripper moves away from the device and the aspirating/dispensing device approaches the device. But the waiting periods may also just serve the purpose to allow incubation of a liquid in the device with reagents in the device or with the device. Or the waiting period may be needed because the particular action as scheduled in the instrument cycle is not needed for the particular device in the particular cycle.

A preferred instrument cycle has a duration of between 5 and 1200, more preferred between 15 and 600, most preferred between 20 and 120 seconds. One instrument cycle may contain between 2 and 60, preferably between 3 and 30, most preferred 4 and 6, shaking periods and about the same number of idle periods. For example, an instrument cycle may start with a shaking period, followed by an idle period. The length of a shaking period may be optimized taking into consideration the number of steps to be performed within an instrument cycle and the time required and sufficient for thorough mixing. The length of the idle periods may be determined taking into consideration the time needed for the particular action to be performed during the idle period. Particularly, if intended to perform more than one action using the same handling device, e.g. the same gripper, the idle period for loading and unloading the device may be required to be longer than in processes in which the actions are performed in subsequent instrument cycles. During the same idle period the treatment station can be loaded or re-loaded with a new device and or washing liquid can be transferred into the chamber of the device. At the end of the dedicated instrument cycle, another instrument cycle will start in the same recess (until the tasks to be performed on the instrument are completed). In this following cycle, any or all of the process steps performed in the first cycle can be performed, in accordance with the requirements of the particular treatment and device, dependent upon the overall process to be performed in the device. In the next cycle, the recess can be loaded with another device. Depending upon the task, the number of cycles to be performed, and the actions performed may differ from the number of cycled for the device before.

Preferably, the treatment station is selected from the group consisting of a washing station, a binding station and a staining station.

In a washing station, a device will be loaded, washing liquid will be added, the washing liquid may be sipped and spitted from and into a pipette tip and the washing liquid may be removed from the device. For complete purification, it may be required to repeat the washing process in the same device with fresh (unused) washing liquid. Thus, in the second cycle, the steps ‘unload transport’ and ‘load transport’ will simply be omitted. During the periods reserved for these actions, there will be no particular actions. All other actions during this cycle will be performed as in the earlier cycle. After performing as many cycles as required the last cycle will comprise the transport of the device into the next treatment station, e.g. the detection station. The washing procedure may include heating the content of the device up to a particular temperature, for which washing is particularly efficient.

In a binding station, the steps loading of the device into the recess, filling reagents, and unloading the device from the recess are preferably performed (filling reagents can even be performed prior to loading the device). During binding, more preferably during hybridization, the liquid contained in the device is preferably heated to a defined temperature useful for binding. This heating may be done at any time, independent form the start of shaking periods and idle periods.

The same combination of steps can be made for the staining station, i.e. combine reacting the surface in the device with reagents for staining and washing the device from superfluous staining reagent. Again, preferably, the liquid is preferably held at a defined temperature optimal for staining.

In a preferred mode of operation the treatment station is used for binding, staining and washing. Obviously, this will need special devices to be capable to access the device in the recess. In addition, appropriate periods for the additional actions sequences, like pipetting of additional reagents during the idle periods on the treatment station are reserved.

A cycle describes the time necessary and a sequence of operations between identical instrument actions, either in on recess or in different recesses, on the same treatment station or on different treatment stations. The cycle considers all possible actions necessary and allows a clocked processing of all assay formats running on an instrument. The cycle is repeated on the instrument for different devices and for each diagnostic test respectively. A cycle is preferably identical in length for all recesses. If different tests with different treatment times are running simultaneously on the instrument the actions performed for each device in each cycle may be different, though. I.e., not all possible actions are performed for each device. The time and thus the number of instrument cycles for which each device is kept within a particular recess may depend upon the particular assay to be performed within said device. The cycle length is given by the process times for device treatments as hybridization, washing and detection, where the processes can last for several cycles. If so, the process stations require several device positions.

Preferably, the method according to the invention comprises performing a second analysis in a second device on the same instrument, wherein at least one of the actions performed on said second device is performed in an instrument cycle different from the instrument cycle in which the action on the first device is performed. Preferably, the analyses require different times to come to completion due to different reaction steps to be performed on the different samples and thus devices. For example, for genotyping analysis, the time required is much smaller than for gene expression analyses. The present invention has the advantage that these analyses can be performed in parallel, i.e. more than one genotyping analysis can be made during the time of one gene expression analysis. In such cases, the instrument cycle will be smaller than the time required for the time needed for the analysis needing the longest time, i.e. the gene expression analysis.

On the other side, it is preferred if the length of instrument cycles for recesses on different treatment stations is the same on one instrument. More preferably, the instrument cycles on different treatment stations are clocked, i.e. start at the same time.

In FIG. 8 there is shown schematically a mode of this embodiment of the invention. It shows the actions performed in 6 different recesses on the same treatment station, exemplary a washing station. Grey bars designate actions and black bars designate controlled incubation while heating and shaking (in intervals). A1 means action 1 (unload), A2 means action 2 (load), A3 means action 3 (liquid transfer) and A4 means action 4 (wash; sip and spit). In the first cycle (cycle 1), device 1 is loaded into recess 1 (for details, like the exact starting time (in seconds) for the loading process within one cycle see FIG. 7). In the second cycle (cycle 2), device 2 is loaded into recess 2 and washing liquid is transferred into device 1 in recess 1. From now on, until the end of cycle 7, there are no actions selected from transfer, loading and unloading being done on device 1 in recess 1 any more. But the device is shaken and temperature controlled as all other devices, thus allowing contaminating components to dissolve from the micro-array surface into the washing liquid. The same treatment is performed on the other devices except devices 5 and 6. Device 5 is placed in recess 5 in cycle 5 and unloaded after actions in cycle 10 after additional washings (see FIG. 7) in cycles 8 and 9. Device 6 is placed in recess 6 in the loading period of cycle 6. However, due to the particular assay to be performed in device 6, the process is finished already in cycle 10, even after performing additional washings in cycles 9 and 10. This example shows that the cycle times for all devices in the different recesses are the same, while the overall treatment time and thus the residence times of the devices in their respective recesses are different. The advantage of the invention to safe time gets very evident when considering the fact that, for example, recess 1 can be reloaded with a new device (device 7) in cycle 8; in the example, it is loaded in cycle 12.

The same procedure can be used in a binding station when replacing the washing liquid by a binding liquid, e.g. by a hybridization liquid.

This process allows conducting processes of different assay formats having different length in the same treatment station.

In particular embodiments, the invention has the advantage of using clocked processes with multiple binding and wash cycles in a freely accessible, preferably heatable treatment station comprising a shaker unit.

### REFERENCE NUMERALS

1 Device
2 Flat carrier (shown from back surface)
3 Cap
4 Locking frame
5 Space for bar code label
6 Drive
7 Gear box
8 Drive belt
9 Eccentric
10 Heater
11 Isolation
12 Device carrier
13 Drive
14 Horizontal carriage
15 Drive
16 Vertical carriage
17 Needle module
18 Valve unit
19 Needle cleaning module
20 Bottle carrier
21 Cooling ducts
22 Second 3-dimensional engagement element
23 First 3-dimensional engagement elements
101 Sample input module
102 Reagent input module
103 Waste disposal position
104 Incubator
105 Device input module
106 Hybridization station
107 Washing station
108 Detection station
109 Staining station
110 Rinsing station
111 Transfer unit
112 Waste unit
113 Computer
114 3-dimensional constructional element of the gripper
115 Recess
201 Shaking period
202 Idle period

1. An instrument for processing one or more analytical devices containing immobilized binding reagents comprising
a sample input station,
one or more treatment stations selected from the group consisting of a binding station, a staining station and a washing station, at least one of said treatment stations comprising a shaker unit comprising a recess for receiving an analytical device, said recess having an upper opening for placing said analytical device into said recess
a detection station, and
a transfer module
wherein said transfer unit comprises a robotic arm comprising a gripper unit horizontally moveable to position said analytical device above said recess and vertically moveable to position said analytical device through said opening in said recess.

2. The instrument according to claim 1, wherein said at least one treatment station is selected from a combined binding and staining, a combined staining and washing, a combined binding and washing and a combined binding, staining and washing station.

3. The instrument according to claim 1, wherein said treatment station is equipped with a heating unit.

4. The instrument according to claim 1, wherein said shaker unit is equipped with a mechanical drive allowing for shaking with a frequency of between 10 and 50 Hz.

5. The instrument according to claim 1, wherein said shaker unit comprises recesses for 2 or more devices.

6. The instrument according to claim 1, wherein said recess comprises 3-dimensional constructional elements for exact vertical and horizontal positioning of said analytical device.

7. The instrument according to claim 1, wherein said shaker unit is equipped with a mechanical drive allowing for shaking with an amplitude of between 0.1 and 10 mm.

8. The instrument according to claim 1, wherein at least one of said treatment station and said transfer module further comprises an aspiration-dispensing unit vertically moveable to introduce a liquid transfer device into an analytical device positioned on said treatment station.

9. The instrument according to claim 1, wherein said shaker unit comprises a drive unit to lead said device on a predetermined path.

10. The instrument according to claim 1, wherein actions of components of said instrument are controlled by a computer program.

11. The instrument according to claim 10, wherein said computer program controls loading and unloading of the device, aspiration and dispensing of liquids, shaking the device and transport of the device within said instrument.

12. The instrument according to claim 10, wherein said computer program induces regular intervals of shaking periods and idle periods in the shaker unit.

13. The instrument according to claim 1, wherein said treatment station is further equipped with a cooling element.

14. The instrument according to claim 1, wherein the transfer module is construed to allow transportation of single analytical devices.

15. A diagnostic system for determining one or more analytes in a sample comprising
an instrument according to claim 1, and
one or more devices containing immobilized binding reagents.

16. The system according to claim 15, wherein
said device comprises first 3-dimensional engagement elements,
said shaker unit comprises a recess for receiving an analytical device, said recess having an upper opening for placing said analytical into said recess, said recess further comprising 3-dimensional constructional elements for exact vertical and horizontal positioning of said analytical device through said first 3-dimensional engagement elements of said device, and
at least one of said treatment station and said transfer module further comprises an aspiration-dispensing unit vertically moveable to introduce a liquid into an analytical device positioned on said treatment station.

17. The system according to claim 15, wherein
said device comprises second 3-dimensional engagement elements, and
said transfer module comprises 3-dimensional constructional elements for gripping said device through said second 3-dimensional engagement elements.

18. The system according to claim 16, wherein the binding reagents are immobilized on a flat surface facing a processing chamber of the device.

19. The system according to claim 15, wherein the binding reagents are nucleic acid probes.

20. The system according to claim 15, wherein the binding reagents are arranged in arrays.

21. The system according to claim 15, wherein the sample has a volume of between 10 and 500 μm.

22. The system of claim 15, further comprising
an analyte purification station.

23. The system of claim 15, further comprising
an amplification station.

24. The system of claim 15, further comprising a data management unit that allows sample tracing from input station to detection result.

25. The system according to claim 16, wherein said device has a cap pierceable by said liquid transfer device.

26. A method for determining one or more analytes in a sample using an instrument comprising the steps
inserting the sample in a sample input device into an input station on said instrument,
inserting one or more devices containing immobilized binding reagents into a disposable input station on said instrument,
starting a controlled, automated procedure to transport the sample through a binding station and a detection station comprising
transferring the sample into one of said devices,
transferring reagents into said device,
transporting said device into said binding station said binding station comprising a shaker unit comprising a recess for receiving an analytical device, said recess having an upper opening for placing said analytical device into said recess,
keeping said device in said binding station under conditions allowing binding of the analyte to be determined with said binding reagents,
removing the liquid from the device,
adding and removing a washing liquid to the device and from the device,
transporting said device into said detection station, and
detecting a signal based on the binding of said analyte to said binding reagents, and
determining the analyte based on said signal.

27. The method according to claim 26, further comprising adding and removing a stain buffer to said device, and adding and removing a washing liquid to the device and from the device.

28. The method according to claim 26, further comprising transporting said device from said binding station to a washing station,

29. The method according to claim 26, wherein the controlled automated procedure comprises removing said device from said detection station.

30. The method according to claim 26, wherein the analyte contains an amplified target sequence.

31. The method according to claim 26, wherein the instrument is an instrument according to claim 1.

32. A method for automated processing of one or more devices containing immobilized binding reagents comprising the steps
providing an instrument comprising one or more treatment stations,
providing one or more devices containing immobilized binding reagents in a disposable input station on said instrument,
providing a sample in a sample input station on said instrument,
starting a controlled, automated procedure to transport the device through said instrument comprising the steps
transferring the sample into said device,
transferring reagents into said device,
transporting said device into said treatment station wherein said treatment station is selected from the group consisting of a binding station, a staining station and a washing station, at least one of said treatment stations comprising a shaker unit comprising a recess for receiving an analytical device, said recess having an upper opening for placing said analytical device into said recess,
maintaining said device in said treatment station under conditions allowing said treatment
wherein said device is shaken during treatment in a recess of a shaker unit of said treatment station, said recess having an upper opening for placing said analytical device into said recess and wherein said transport into said treatment station is done by a transfer unit comprising a robotic arm comprising a gripper unit horizontally moveable to position said analytical device above said recess and vertically moveable to position said analytical device through said opening in said recess.

33. The method according to claim 32, wherein said treatment is a binding process.

34. The method according to claim 32, wherein said treatment is a staining process.

35. The method according to claim 32, wherein said treatment is a washing process.

36. The method according to claim 32, wherein said conditions comprise heating said device.

37. The method according to claim 32, wherein said shaking is done with a frequency of between 10 and 50 Hz.

38. The method according to claim 32, wherein 2 or more devices are shaken in parallel.

39. The method according to claim 32, wherein said shaking is done with an amplitude of between 0.1 and 10 mm.

40. The method according to claim 32, wherein said method is controlled by a computer program.

41. A method for performing an analysis in a device using an instrument comprising treating said device in said instrument during at least two shaking periods and at least two idle periods by performing at least two actions selected from the group of
loading said device into a recess on a treatment station on said instrument,
transferring a liquid into said device when located in a recess on a treatment station on said instrument,
unloading said device from a recess on a treatment station on said instrument, and
washing said device in a recess on a treatment station on said instrument, wherein said actions are performed on the device in the same recess at fixed and non-overlapping action periods within repetitive instrument cycles of substantially the same length, said action periods not overlapping with said shaking periods.

42. The method of claim 41 wherein said treatment station is selected from the group consisting of a washing station, a binding station and a staining station.

43. The method according to claim 41, further comprising performing a second analysis in a second device on the same instrument, wherein at least one of the actions performed on said second device is performed in an instrument cycle different from the instrument cycle in which the action on the first device is performed.

44. The method according to claim 41, further comprising treating said device in a recess on another treatment station having an instrument cycle of substantially the same length.

45. The method according to claim 41, wherein the instrument cycles on different treatment stations are clocked.
